(19) United States
(12) Patent Application Publication
(10) Pub. No.: US 2009/0192125 A1
(43) Pub. Date: Jul. 30, 2009

(54) Local anti-infective agent for treatment of nail fungal infections

(75) Inventors: David M. Cohen, Lauderdale By The Sea, FL (US); Eugene R. Cooper, Berwyn, PA (US)

Correspondence Address: ROPES & GRAY LLP, PATENT DOCKETING 39/41, ONE INTERNATIONAL PLACE, BOSTON, MA, 02110-2624, US

(73) Assignee: DermWorx Incorporated, Hollywood, FL (US)

(21) Appl. No.: 12/228,675

(22) Filed: Aug. 13, 2008

Related U.S. Application Data
(63) Continuation-in-part of application No. 12/028,188, filed on Feb. 8, 2008.
(60) Provisional application No. 60/888,825, filed on Feb. 8, 2007.

(51) Int. Cl. A61K 31/60; A61K 31/20; A61K 31/19; A61K 31/192; A61K 31/4425; C07D 213/70; A61K 31/198; A61K 31/135; A61P 31/10

(52) U.S. Cl. 514/162; 514/558; 514/557; 514/159; 514/568; 514/345; 546/290; 514/562; 514/649

(57) ABSTRACT

A topical composition for treating nail fungal infections that utilizes an acidic antifungal agent with a molecular weight no greater than 170 Daltons in a formulation having a pH less than or equal to the pKa of the acidic antifungal agent plus one. Possible antifungal agents include omadine, octanoic acid, sorbic acid, hexanoic acid, and benzoic acid. The antifungal agent can be combined with a delivery system such as a lacquer, a gel, a patch, or a hydrating system. A second therapeutic agent such as a 5-fluorocystine or terbinafine can be included.

### RELATED APPLICATIONS

This application is a continuation-in-part of and claims priority to U.S. application Ser. No. 12/028,188, filed Feb. 8, 2008, which claims the benefit of priority to U.S. provisional application Ser. No. 60/888,825, filed Feb. 8, 2007. The disclosures of each of the foregoing applications are hereby incorporated by reference in their entirety.

### BACKGROUND

Nail infections can be caused by a number of agents, many known, but some unknown at this time. Agents known to cause infections include molds, fungi, and yeast organisms. Onychomycosis is the most common nail infection. A fungal infection, onychomycosis can be caused by C. albicans, C. parappsilosis, or Scopulariopsis brevicaulis. Other etiologic agents known to cause onychomycosis include Aspergillus flavus, A. candidus, A. fumigatus, A. sydowi, A. terreus, and other Aspergillus species, Cephalosporum species, and Fusrium oxysporum. Onychomycosis is associated with chronic paronychia where active invasion of the nail plate is less frequent. Paronychia can be identified by painful, red swelling around the nail, often at the cuticle or at the site of a hangnail or other injury. With fungal infections, symptoms of paronychia often develop slowly over time. Tinea unguium, on the other hand, is an invasive disease of the nail plate caused by a dermatophyte, most commonly T. interdigitale, T. rubrum, or T. metagrophytes. Generally, tinea ungium is classified into two subtypes—leukonychia mycotica (superficial white onychomycosis, SWO) and invasive sublingual onychomycosis (commonly called ringworm of the nail). In leukonychia mycotica, the nail is invaded from the top exhibiting pitting or infected patches on the surface of the nail. Additionally, white spots or streaks on the nail surface are apparent, as well as a soft or powdery nail surface. Other symptoms may include damaged, brittle, brown or gray nail surfaces. This type of infection is almost always produced by T. mentagrophytes.

Onychomycosis has long been recognized as one of the most difficult fungal infections to treat. The lengthy period over which the nail takes to grow, the hardness of the nail plate, the location of the infectious process between the nail bed and plate are major factors for difficulty in treatment. Current treatments for these conditions are primarily oral anti-infective agents that are lipophilic and have very large molecular weights and thus would have poor nail penetration if applied topically. Examples of these agents include terbinafine, itraconazole, and fluconazole. Treatments with these agents can last for months and are associated with systemic toxicity. In order to avoid systemic toxicities, it is important to develop methods to treat these local infections using local anti-infective agents. Such treatments can provide significant improvements to current therapies including less toxicity and potentially greater efficacy.

One currently commercially-available topical treatment of onychomycosis is ciclopirox. Ciclopirox has only been marginally successful and it has been shown to have poor penetration across the layers of the nail. The nail, a highly keratinized membrane, is a formidable barrier to diffusion, and the approach has been to utilize existing oral antifungal drugs in topical formulations so that they might penetrate the nail, but this approach has not worked well. Accordingly, there remains a need for improved treatments of nail fungal infections.

### SUMMARY OF INVENTION

The present invention provides a composition for topical administration of an antifungal agent comprising an acidic antifungal agent with a molecular weight no greater than about 170 Daltons and/or a pharmaceutically acceptable salt thereof in an aqueous formulation having a pH no more than the pKa of the acidic antifungal agent plus about 2.5, such as having a pH no more than the pKa of the acidic antifungal agent plus about two, or even one.

The present invention further provides a composition for topical administration of an antifungal agent comprising an acidic antifungal agent with a molecular weight no greater than about 170 Daltons in a substantially nonaqueous formulation, wherein the acidic antifungal agent optionally comprises a combination of the acidic antifungal agent and a pharmaceutically acceptable salt thereof. In certain such embodiments, the weight ratio of the pharmaceutically acceptable salt of the acidic antifungal agent to the acidic antifungal agent does not exceed 100:1. In certain embodiments, the pharmaceutically acceptable salt of the acidic antifungal agent comprises an ammonium salt of omadine. In certain embodiments, the composition for topical administration of an antifungal agent is for the treatment of a nail fungal infection, such as onychomycosis.

The present invention provides compositions comprising an antifungal agent,

wherein the antifungal agent comprises an ammonium salt of omadine, wherein the ammonium salt of omadine can be not only the NH₄+ salt, but also the corresponding alkyl, dialkyl, trialkyl or tetra-alkyl ammonium salt.

### DETAILED DESCRIPTION OF THE INVENTION

The present invention provides a composition comprising an acidic antifungal agent with a molecular weight no greater than about 170 Daltons and/or a pharmaceutically acceptable salt thereof in an aqueous formulation having a pH no more than the pKa of the acidic antifungal agent plus about 2.5, such as a pH no more than the pKa of the acidic antifungal agent plus about two, or even one.

The present invention provides a composition comprising an acidic antifungal agent with a molecular weight no greater than about 170 Daltons in a substantially nonaqueous formulation, wherein the acidic antifungal agent optionally comprises a combination of the acidic antifungal agent and a pharmaceutically acceptable salt thereof. In certain such embodiments, the weight ratio of the pharmaceutically acceptable salt of the acidic antifungal agent to the acidic antifungal agent does not exceed 100:1. In certain embodiments, the acidic antifungal agent or combination of the acidic antifungal agent and a pharmaceutically acceptable salt thereof, is fully soluble in the nonaqueous formulation. In certain embodiments, the pharmaceutically acceptable salt of the acidic antifungal agent comprises an ammonium salt of omadine. As used herein, the term “substantially nonaqueous formulation” refers to a formulation comprising less than 10% by weight of water, such as less than 9%, 8%, 7%, 6%, 5%, 4%, 3%, 2% or 1% by weight of water.

In certain such embodiments, the composition of the present invention is for topical administration, such as topical administration to a nail surface. For example, the present invention provides a composition for topical administration of an antifungal agent comprising an acidic antifungal agent with a molecular weight no greater than about 170 Daltons and/or a pharmaceutically acceptable salt thereof in an aqueous formulation having a pH no more than the pKa of the acidic antifungal agent plus about 2.5, such as having a pH no more than the pKa of the acidic antifungal agent plus about two, or even one.

The present invention further provides a composition for topical administration of an antifungal agent comprising an acidic antifungal agent with a molecular weight no greater than about 170 Daltons in a substantially nonaqueous formulation, wherein the acidic antifungal agent optionally comprises a combination of the acidic antifungal agent and a pharmaceutically acceptable salt thereof. In certain such embodiments, the weight ratio of the pharmaceutically acceptable salt of the acidic antifungal agent to the acidic antifungal agent does not exceed 100:1. In certain embodiments, the pharmaceutically acceptable salt of the acidic antifungal agent comprises an ammonium salt of omadine. In certain embodiments, the composition for topical administration of an antifungal agent is for the treatment of a nail fungal infection, such as onychomycosis.

In certain embodiments, the acidic antifungal agent is selected from any suitable acidic antifungal agent with a molecular weight no greater than about 170 Daltons. Examples of acidic antifungal agents suitable for use in the compositions of the present invention include omadine, octanoic acid, sorbic acid, hexanoic acid, salicylic acid, and benzoic acid. In certain embodiments, the acidic antifungal agent comprises omadine. In certain embodiments, compositions of the present invention comprise pharmaceutically acceptable salts of an acidic antifungal agent. Exemplary salts of omadine include sodium omadine, potassium omadine, and ammonium omadine, wherein ammonium salts of omadine include not only the NH₄+ salt, but also the corresponding alkyl, dialkyl, trialkyl or tetra-alkyl ammonium salts. In certain embodiments, the composition according to the present invention comprises more than one antifungal agent (e.g., more than one of the antifungal agents listed above). In certain embodiments of the composition of the present invention, the acidic antifungal agent comprises a combination of more than one acidic antifungal agent listed above. In certain such embodiments, the acidic antifungal agent comprises a combination of omadine and benzoic acid.

In certain embodiments, the acidic antifungal agent has a solubility in water above 0.1 mg/mL at 25° C., such as above 0.5 mg/mL at 25° C., or even above 1.0 mg/mL at 25° C.

In certain embodiments, the acidic antifungal agent (e.g., any one of the acidic antifungal agents listed above) and/or pharmaceutically acceptable salt thereof comprises from 0.1 to 30 percent by weight of the composition, such as from 0.1 to 20 percent by weight of the composition. In certain embodiments, the acidic antifungal agent comprises from one to six percent by weight of the composition. In certain embodiments, the acidic antifungal agent comprises omadine and comprises from one to six percent by weight of the composition.

In certain embodiments, the pH of the composition is about equal to the pKa of the acidic antifungal agent. The pKa is the negative log of the acid dissociation constant. As used herein, the term “equal to the pKa” is meant to mean that the pH is equal to the pKa±0.1. By being in a pH equal to the pKa, at least about 50 percent of the acidic antifungal agent is in its free acid form in the solution. In certain embodiments, the pH of the formulation is less than or equal to about the pKa. In other embodiments, the pH of the composition for treatment of a fungal infection is no less than the pKa of the acidic antifungal agent minus about one. In certain embodiments, the pH of the formulation is no more than the pKa plus 2.5, such as no more than the pKa plus two, or even one. By being in a pH equal to the pKa plus one, 30 to 50% of the acidic antifungal agent is in its free acid form in solution.

In certain embodiments, the composition of the present invention further comprises a glycol, such as an alkane diol, a polyethylene glycol, or a polyol, such as glycerol. In certain such embodiments, the alkane diol is selected from ethylene glycol, propylene glycol or a butane diol, such as butane-1,2-diol, butane-2,3-diol, or butane-1,4-diol. In certain embodiments, the glycol is not ethylene glycol. In certain embodiments, the alkane diol comprises propylene glycol. In certain embodiments, the composition of the present invention further comprises glycerol or propylene glycol. In certain embodiments, the glycol, such as an alkane diol, polyethylene glycol, or polyol, such as glycerol, comprises from 0 to 65% by weight of the composition, such as from 10% to 65% by weight of the composition, such as from 20% to 60% by weight of the composition.

In certain embodiments, the composition of the present invention further comprises a film forming polymer. In certain such embodiments, the film forming polymer is selected from any suitable film forming polymer. Examples of suitable film forming polymers for use in the compositions of the present invention include polyvinyl alcohol, polyvinylpyrrolidone, cellulose, and cellulose derivatives, such as polymers comprising carboxymethyl-, hydroxyethyl-, hydroxypropyl-, hydroxypropylmethyl- and methyl-cellulose. In certain embodiments, the composition of the present invention further comprises hydroxypropylcellulose. Pharmaceutical-grade hydroxypropylcellulose is commercially-available in a variety of molecular weights, any of which would be a suitable in the formulation of the current invention. In certain embodiments, the film forming polymer is initially dissolved in an organic solvent. In other embodiments, the film forming polymer in the formulation for topical application is initially dissolved in an aqueous solvent. In certain embodiments, the film forming polymer (e.g., any one of the film forming polymers listed above) comprises from 0 to 10 percent by weight of the composition, such as from 0 to 5% or 0 to 3% by weight of the composition. In certain embodiments, the film forming polymer (e.g., any one of the film forming polymers listed above) comprises less than 2% by weight of the composition.

In certain embodiments, the composition of the present invention further comprises a volatile solvent. In certain embodiments, the volatile solvent comprises one or more short-chain alcohols and/or water. In certain embodiments, the short-chain alcohol is selected from the isomers of butanol, isomers of propanol, ethanol, and methanol. Alternatively, any other short-chain alcohol safe for topical administration would be appropriate. In certain embodiments, the volatile solvent comprises a short-chain alcohol other than methanol. In certain such embodiments, the volatile solvent comprises ethanol, propanol, isopropanol, butanol, or butan-2-ol. In certain embodiments, the volatile solvent comprises ethanol or isopropanol. In certain embodiments, the volatile solvent comprises water. In certain embodiments, the volatile solvent comprises a combination of one or more volatile solvents, such as a combination of one or more short-chain alcohols or a combination of one or more short-chain alcohols and water. In certain such embodiments, the volatile solvent comprises a combination of one or more volatile solvents selected from ethanol, propanol, isopropanol, butanol, butan-2-ol, and water. In certain embodiments, the volatile solvent comprises a combination of isopropanol and water. In certain embodiments, the volatile solvent comprises from 20% to 65% by weight of the composition, such as from 30% to 60% by weight of the composition.

The present invention provides a composition for topical administration of an antifungal agent comprising an acidic antifungal agent with a molecular weight no greater than about 170 Daltons and/or a pharmaceutically acceptable salt thereof; a glycol, such as an alkane diol, a polyethylene glycol, or a polyol, such as glycerol; and a volatile solvent, such as one or more short-chain alcohols and/or water. In certain embodiments, the acidic antifungal agent with a molecular weight no greater than about 170 Daltons comprises omadine. In certain embodiments, the glycol comprises propylene glycol. In certain embodiments, the volatile solvent comprises isopropanol and/or water. In certain embodiments wherein the volatile solvent is an aqueous volatile solvent (e.g., the volatile solvent comprises greater than or equal to 10% by weight of water), the composition has a pH no more than the pKa of the acidic antifungal agent plus about 2.5, such as a pH no more than the pKa of the acidic antifungal agent plus about two, or even one. In certain embodiments, the composition further comprises a film forming polymer, such as hydroxypropylcellulose. In certain embodiments wherein the volatile solvent is a non-aqueous volatile solvent (e.g., the volatile solvent comprises less than 10% by weight of water), the pharmaceutically acceptable salt of the acidic antifungal agent is an ammonium salt of omadine. In certain embodiments wherein the volatile solvent is a non-aqueous volatile solvent (e.g., the volatile solvent comprises less than 10% by weight of water), the weight ratio of the pharmaceutically acceptable salt of the acidic antifungal agent to the acidic antifungal agent does not exceed 100:1. In certain embodiments, the composition further comprises a film forming polymer, such as hydroxypropylcellulose. In certain such embodiments, the composition comprises less than 2% by weight of the film-forming polymer.

For example, the present invention provides a composition for topical administration of an antifungal agent, comprising omadine and/or an ammonium salt thereof, propylene glycol, and a volatile solvent such as isopropanol, ethanol, and/or water. In certain such embodiments wherein the volatile solvent is an aqueous volatile solvent (e.g., the volatile solvent comprises greater than or equal to 10% by weight of water), the pH of the composition is no more than the pKa of the omadine plus about 2.5. In certain embodiments, the composition further comprises a film forming polymer, such as hydroxypropylcellulose.

The present invention provides compositions comprising an antifungal agent, wherein the antifungal agent comprises an ammonium salt of omadine, wherein the ammonium salt of omadine can be not only the NH₄+ salt, but also the corresponding alkyl, dialkyl, trialkyl or tetra-alkyl ammonium salt.

The present invention provides a method of delivering a composition of the present invention to an infected surface, such as a nail surface and/or surrounding tissue. In certain embodiments, the nail surface comprises a toenail. In certain embodiments, the surface is infected with a nail fungus, such as onychomycosis. In certain embodiments, the compositions of the present invention are formulated for delivery in the form of a lacquer, a gel, a patch, or a hydrating system.

The present invention also provides a method for administering a composition of the present invention to an animal in need of such treatment, comprising applying an effective amount of the composition to an infected nail surface and/or surrounding tissue of the animal. In certain embodiments, the nail surface comprises a toenail. In certain embodiments, the nail surface and/or surrounding tissue is infected with a nail fungus, such as onychomycosis. In certain embodiments, the animal is a human.

The present invention further provides a method for providing an antifungal effect in an animal, comprising administering to a nail surface and/or surrounding tissue of an animal in need of such treatment a therapeutically effective amount of a composition according to the present invention. In certain embodiments, the nail surface comprises a toenail. In certain embodiments, the nail surface is infected with a nail fungus, such as onychomycosis. In certain embodiments, the animal is a human.

The present invention provides a method of treating onychomycosis, comprising administering to a nail surface and/or surrounding tissue infected by onychomycosis a therapeutically effective amount of a composition as described herein. In certain embodiments, the nail surface comprises a toenail. For example, the present invention provides a method of treating-onychomycosis, comprising administering to a nail surface and/or surrounding tissue in need of such treatment a therapeutically effective amount of a composition of the present invention.

In certain embodiments, the compositions of the present invention provide enhanced topical delivery of antifungal agents for the treatment of nail infections, also called onychomycosis or tinea unguium (also sometimes called ringworm of the nail). The causative agents for these conditions are a series of infective agents which encompass dermatophytes, fungal agents, yeast, or non-dermatophyte molds. In certain embodiments, compositions of the present invention comprise small molecular weight substances with particular characteristics and particular formulations for the topical treatment of these conditions.

The present invention provides a topical therapy comprising antifungal agents that can pass across the nail based on their molecular properties. The compositions of the present invention ensure the penetration of these agents across the multiple layers that form the nail. The nail barrier is very sensitive to the size (and hence the molecular weight) of the diffusing molecule. In fact, nail diffusion is so selective for molecular weight that agents with molecular weight preferably below about 170 Daltons, but not limited to such, are the best choice for topical treatment of onychomycosis. One aspect of the invention encompasses using any low molecular weight (e.g., less than about 170 Daltons) acidic compounds that are generally known anti-fungal agents as effective agents to treat nail-related fungal infections.

In certain embodiments, the low molecular weight acidic antifungal agents have a solubility in water above 1 mg/mL, but are not limited as such. Combinations of agents with the properties discussed or with other existing therapies would also be effective for treating onychomycosis. The agents described can be formulated, but not limited to, as films, lacquers, gels, patches, hydrating systems, etc. for topical delivery and can be dosed in concentrations of 50% and below but not necessarily limited as such. They can be incorporated into polymer films and applied directly to the nail. For cosmetic purposes, agents may be added to mask the unpleasant appearance of the infected nail. These films can be similar to traditional fingernail films used to color the nail. They can contain any type of film forming polymer such as celluloses or cellulosic derivatives and can be formed from organic solvents and/or solvents containing water.

In certain embodiments, the composition of the present invention is formulated for topical administration in the form of a lacquer, gel, patch, film forming system, or hydrating system. In certain embodiments wherein the composition of the present invention is formulated in the form of a patch, the adhesive of the patch comprises the composition of the present invention.

The present invention provides a patch for topical application (e.g., to an infected area), comprising an adhesive comprising a composition of the present invention (e.g., a composition as described above). In certain embodiments, the acidic antifungal agent and/or pharmaceutically acceptable salt thereof, as defined in the compositions of the present invention (e.g., the compositions as defined above) comprises from 0.5 to 20% by weight of the adhesive.

The present invention provides a method of applying a patch as described herein to an infected area, such as an infected nail surface (e.g., a toenail with a fungal infection, such as onychomycosis).

The term “volatile solvent” as used herein refers to a solvent (e.g., a solvent or combination of solvents) that changes readily from solid or liquid to a vapor, e.g., that evaporates readily at some temperature at or below body temperature and less readily at room temperature, such as a solvent that evaporates rapidly between 21 and 37° C. at atmospheric pressure.

The term “healthcare providers” refers to individuals or organizations that provide healthcare services to a person, community, etc. Examples of “healthcare providers” include doctors, hospitals, continuing care retirement communities, skilled nursing facilities, subacute care facilities, clinics, multispecialty clinics, freestanding ambulatory centers, home health agencies, and HMO's.

The term “treating” refers to: preventing a disease, disorder or condition from occurring in a cell, a tissue, a system, animal or human which may be predisposed to the disease, disorder and/or condition but has not yet been diagnosed as having it; stabilizing a disease, disorder or condition, i.e., arresting its development; and relieving one or more symptoms of the disease, disorder or condition, i.e., causing regression of the disease, disorder and/or condition.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

The compositions and methods of the present invention may be utilized to treat an individual in need thereof. In certain embodiments, the individual is a mammal such as a human, or a non-human mammal

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The compositions of the present invention can be administered to a subject topically, for example, as a gel, foam, solution, lotion, cream, ointment, spray, or patch applied to the nail surface and/or surrounding skin. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The compositions of the present invention may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the antifungal agent which produces an antifungal effect.

Besides the components outlined above, the compositions of the present invention can also include adjuvants such as wetting agents, emulsifying and suspending agents, sweetening, flavoring, coloring, perfuming and preservative agents.

Compositions of the present invention include powders, sprays, ointments, pastes, creams, lotions, gels, solutions, and patches. The antifungal agent may be mixed under sterile conditions with the other components of the composition, and with any preservatives, buffers, or propellants that may be required.

The ointments, pastes, creams and gels may contain, in addition to the antifungal agent, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Sprays can contain, in addition to the antifungal agent, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The compositions of the present invention may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like.

Actual dosage levels of the active ingredients in the compositions of the present invention may be varied so as to obtain an amount of the active ingredient that is effective to achieve the desired antifungal response for a particular patient, composition, and mode of administration, without being toxic to the patient.

The selected dosage level will depend upon a variety of factors including the activity of the particular antifungal agent or combination of antifungal agents employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the composition required. For example, the physician or veterinarian could start doses of the composition or antifungal agent at levels lower than that required in order to achieve the desired antifungal effect and gradually increase the dosage until the desired effect is achieved. By “therapeutically effective amount” is meant the concentration of an antifungal agent that is sufficient to elicit the desired antifungal effect. It is generally understood that the effective amount of the antifungal agent will vary according to the weight, sex, age, and medical history of the subject. Other factors which influence the effective amount may include, but are not limited to, the severity of the patient's condition, the disorder being treated, the stability of the antifungal agent, and, if desired, another type of antifungal agent being administered with the composition of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active antifungal agent used in the compositions and methods of the invention will be that amount of the antifungal agent that is the lowest dose effective to produce an antifungal effect. Such an effective dose will generally depend upon the factors described above.

If desired, the effective daily dose of the active antifungal agent (e.g., the compositions of the present invention) may be administered as one, two, three, four, five, six or more sub-doses administered separately at appropriate intervals throughout the day, optionally, in unit dosage forms. In certain embodiments of the present invention, the active antifungal agent (e.g., the compositions of the present invention) may be administered two or three times daily. In further embodiments, the active antifungal agent (e.g., the compositions of the present invention) will be administered once daily.

The patient receiving this treatment is any animal in need, including primates, in particular humans, and other mammals such as equines, cattle, swine and sheep; and poultry and pets in general.

In certain embodiments, the compositions of the present invention may optionally be administered conjointly with another therapeutic agent, such as another antifungal agent. As used herein, the phrase “conjoint administration” refers to any form of administration of two or more different antifungal compounds such that the second compound is administered while the previously administered antifungal compound is still effective in the body (e.g., the two compounds are simultaneously effective in the patient, which may include synergistic effects of the two compounds or compositions). For example, the different antifungal compounds can be administered either in the same composition or in a separate formulation, either concomitantly or sequentially. Thus, an individual who receives such treatment can benefit from a combined effect of different antifungal compounds.

Compositions of the present invention may be conjointly administered with any suitable antifungal compound, including the acidic antifungal compounds listed above. Further exemplary antifungal compounds that may be administered conjointly with compositions of the present invention include 5-fluorocystine, amorolfine, isoconazole, clotrimazole, econazole, econazole nitrate, miconazole, nystatin, terbinafine, bifonazole, amphotericin, griseofulvin, ketoconazole, fluconazole and flucytosine, fezatione, ticlatone, tolnaftate, triacetin, butenafine, butoconazole, butoconazole nitrate, clioquinol, itraconazole, lanoconazole, neticonazole, tioconazole, terconazole, ciclopirox olamine, or triclosan. In certain embodiments, compositions of the present invention may be conjointly administered with 5-fluorocystine or terbinafine.

This invention includes the use of pharmaceutically acceptable salts of the antifungal compounds listed above. In certain embodiments, contemplated salts of the invention include ammonium salts, wherein the ammonium salt can be not only the NH₄+ salt, but also the corresponding alkyl, dialkyl, trialkyl or tetra-alkyl ammonium salts. In certain embodiments, contemplated salts of the invention include Na, Ca, K, Mg, Zn or other metal salts.

The pharmaceutically acceptable acid addition salts can also exist as various solvates, such as with water, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate, as well as coloring agents, release agents, and perfuming agents, preservatives and antioxidants can also be present in the compositions of the present invention.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

The present invention provides a kit comprising:
a) a composition as described herein (e.g., a composition for topical administration of an antifungal agent, such as a composition described above); and
b) instructions for the administration of the composition to an individual in need thereof.

For example, the present invention provides a kit comprising:
a) a composition for topical administration of an antifungal agent comprising an acidic antifungal agent with a molecular weight no greater than about 170 Daltons and/or a pharmaceutically acceptable salt thereof in an aqueous formulation having a pH no more than the pKa of the acidic antifungal agent plus about 2.5, such as having a pH no more than the pKa of the acidic antifungal agent plus about two, or even one; and
b) instructions for the administration of the composition to an individual in need thereof.

The present invention further provides a kit comprising:
a) a composition for topical administration of an antifungal agent comprising an acidic antifungal agent with a molecular weight no greater than about 170 Daltons in a substantially nonaqueous formulation, wherein the acidic antifungal agent optionally comprises a combination of the acidic antifungal agent and a pharmaceutically acceptable salt thereof, wherein the weight ratio of the pharmaceutically acceptable salt of the acidic antifungal agent to the acidic antifungal agent optionally does not exceed 100:1, and wherein the pharmaceutically acceptable salt of the acidic antifungal agent optionally comprises an ammonium salt of omadine; and
b) instructions for the administration of the composition to an individual in need thereof.

The present invention provides a kit comprising:
a) a composition for topical administration of an antifungal agent, comprising omadine and/or a pharmaceutically acceptable salt thereof, a film forming polymer, a glycol or glycerol, and an aqueous volatile solvent, wherein the pH of the composition is no more than the pKa of the omadine plus about 2.5 r; and
b) instructions for the administration of the composition to an individual in need thereof.

In certain embodiments, the kit further comprises instructions for the administration of the composition conjointly with another antifungal agent. In certain embodiments, the kit further comprises a second pharmaceutical formulation, including but not limited to a composition according to the present invention comprising a second antifungal agent.

The present invention provides a kit comprising:
a) a pharmaceutical formulation or one or more single dosages forms each comprising an antifungal agent, such as any of the antifungal agents listed above; and
b) instructions for the administration of the pharmaceutical formulation or one or more single dosage forms conjointly with a composition of the present invention.

In certain embodiments, the invention relates to a method for conducting a pharmaceutical business, by manufacturing a composition of the present invention, or a kit as described herein, and marketing to healthcare providers the benefits of using the composition or kit for the treatment of an individual in need thereof.

In certain embodiments, the invention relates to a method for conducting a pharmaceutical business, by providing a distribution network for selling a composition of the present invention, or kit as described herein, and providing instruction material to patients or physicians for using the composition or kit for the treatment of an individual in need thereof.

In certain embodiments, the invention comprises a method for conducting a pharmaceutical business, by determining an appropriate composition of the present invention comprising an appropriate dosage of an antifungal agent for the treatment of an individual in need thereof, conducting therapeutic profiling of identified compositions for efficacy and toxicity in animals, and providing a distribution network for selling an identified composition as having an acceptable therapeutic profile. In certain embodiments, the method further includes providing a sales group for marketing the composition to healthcare providers.

In certain embodiments, the invention relates to a method for conducting a pharmaceutical business by determining an appropriate composition of the present invention comprising an appropriate dosage of an antifungal agent for the treatment of an individual in need thereof, and licensing, to a third party, the rights for further development and sale of the formulation.

### EXEMPLIFICATION

### Example 1

The following describes examples of formulations containing omadine or fatty acids in solutions or dispersions containing organic solvents, a polymer, and water at pH equal to or below the pKa of the acidic antifungal agents. All percentages used in the application are weight percentages unless otherwise noted.

### Formulation 1:

A formulation was prepared by mixing the following components:

10% omadine at pH 4.7;

isopropyl alcohol; and

hydroxypropylcellulose

to produce a final concentration of omadine of 1%.

### Formulation 2:

A formulation was prepared by mixing the following components:

10% omadine at pH 4.7;

isopropyl alcohol; and

hydroxypropylcellulose

to produce a final concentration of omadine of 2.5%.

### Formulation 3:

A formulation was prepared by mixing the following components:

hexanoic acid dispersed in water at pH 3; and

3% hydroxypropylcellulose

to produce 1% hexanoic acid dispersed in the gel.

### Example 2

These studies measured the penetration of stock solutions of sodium omadine in human fingernails. Sodium omadine (NaOM) 98% was obtained from Acros Organics, a division of Global Fisher Scientific.

Stock Solution #1. Dissolved 100 mg of NaOM in 100 mL of purified water (1.0 mg/mL).

Stock Solution #2. Diluted 2 mL of Stock Solution #1 to 100 mL of purified water (20 μg/mL).

Standard Solution #1 (2.50 μg/mL). Diluted 12.5 mL of Stock Solution #2 to 100 mL with purified water.

Standard Solution #2 (5.00 μg/mL). Diluted 25 mL of Stock Solution #2 to 100 mL with purified water.

Standard Solution #3 (7.50 μg/mL). Diluted 37.5 mL of Stock Solution #2 to 100 mL with purified water.

Standard Solution #4 (10.0 μg/mL). Diluted 50 mL of Stock Solution #2 to 100 mL with purified water.

Standard Solution #5 (20.0 μg/mL). No dilution was necessary, same as Stock Solution #2.

Nail clips, pooled from six human volunteers were cut into small pieces of approximately 0.5-1 mm×2-3 mm. The nail pieces, 0.1 g, were placed in each of the above solutions at room temperature and incubated for twenty-four hours. After incubation, the pieces were washed quickly three times with purified water to remove any residual solution on the nail surface. The pieces were then incubated in 1 mL of purified water for twenty-four hours at room temperature to extract the NaOM absorbed into the nail bed (“Extracting Solution”).

A standard curve of NaOM was made by measuring the absorption at 318 nm for a 500 μL sample containing 0.25, 0.50, 0.75, 1.0, and 2.0 μg of NaOM. An aliquot (50 μL) of each Standard Solution (#1-#5) was diluted to 500 μL with 1 mM cupric chloride (CuCl₂). The standard solutions were freshly prepared and incubated at room temperature for fifteen minutes prior to spectrophotometric determination.

The amount of NaOM in each sample of Extracting Solution was determined at 318 nm. Similarly, a 50 μL aliquot of each Extracting Solution was diluted to 500 μL with 1 mM CuCl₂ and incubated at room temperature for 15 minutes. The amount of NaOM in each sample of Extracting Solution was then determined at 318 nm by spectrophotometry. The concentration of the NaOM adsorbed into the nail was determined from the standard curve.

The standard curve at concentrations between 0.25-5 μg/500 μL was linear. The amount of NaOM in each sample was calculated and is presented in Table 1.

TABLE 1

| | NaOM in 50 μL | NaOM conc in | NaOM in 0.1 g |
|---|---|---|---|
| Wk Sol Conc | sample | sample | nail |
| 0.1 mg/mL | 1.33 μg | 27 μg/mL | 3.99 μg |
| 0.05 mg/mL | 0.70 μg | 14 μg/mL | 2.10 μg |
| 0.01 mg/mL | 0.34 μg | 7 μg/mL | 1.02 μg |

### Example 3

These studies measured the penetration of stock solutions of hydroxypyridine-2-thione zinc (PT) in human fingernails.

Nail clips, pooled from six volunteers, were minced into small pieces of approximately 0.5-1 mm×2-3 mm. PT was dissolved in 100% DMSO at a concentration of 2 mg/mL to form a PT stock solution. PT working solution was made by mixing 300 μL of the PT stock solution with 700 μL of water. The final concentration was 0.6 mg/mL (30% DMSO). The nail pieces (0.1 g) were incubated in 1 mL of PT working solution overnight at room temperature. After incubation, the pieces were washed with water three times in a spin column. The pieces were then incubated in 200 μL of 30% DMSO to extract PT for 4 hours at room temperature (“Extracting Solution”).

A standard curve of PT was established by measuring the absorption at 318 nm in 500 μL samples of 30% DMSO containing 0.25, 0.5, 0.75, 1, and 2 μg PT. Standard solutions and subsequent dilutions were prepared as above. The solutions in their desired concentrations were then incubated at room temperature for 5 min prior to spectrophotometric determination.

Similarly, a 50 μL aliquot of the Extracting Solution was diluted to 500 μL with 1 mM CuCl₂. The amount of PT in the sample of Extracting Solution was then determined at 318 nm by spectrophotometry. The concentration of the PT adsorbed into the nail was determined from the standard curve.

The following results were obtained. The standard curve at the concentrations between 0.25-2 μg/500 μL was linear. The concentration of PT was calculated to be 1.15 μg/50 μL, or 23 μg/mL.

The following conclusion was made based on the results. There is significant penetration of hydroxypyridine-2-thione zinc into fingernails after twelve hours or less incubation time.

### Example 4

### Determination of the in vitro Nail Penetration of Five Omadine Formulations using the Franz Human Nail Finite Dose Model

### Abbreviations:

C Centrigrade/Celcius

cm centimeter

CPM Counts per minute

ddH₂O Distilled De-ionized water

DPM Decays per minute

equ equivalents

³H₂O tritiated water

HPLC High Performance Liquid Chromatograpy

hr hour

mL milliliter

MS Mass Spectrometer

ng nanogram

PBS Phosphate Buffered Saline

SD Standard Deviation

SE Standard Error

SLS Sodium Lauryl Sulfate

μg microgram

μ micron

μL microliter

w/w weight to weight

w/v weight to volume

The study was designed to evaluate the human fingernail absorption pharmacokinetics of Omadine. Absorption was measured in human ex vivo human fingernails, in vitro, using the finite dose technique and modified Franz Diffusion Cells.

The products were tested in duplicate on fingernails from three different cadaver donors per formulation for the fingernail absorption of Omadine over a 14-day dose period. The fingernails were dosed once per day. After the last sample (day 15), the fingernails were gently washed with soap (1% SLS in water), and water rinsed, using cotton tipped swabs. Receptor solution samples were collected once per day (24±3 hour intervals) prior to each day's subsequent dose application through to day 15. The samples were analyzed for Omadine content by High Performance Liquid Chromatography (HPLC).

### Introduction:

The in vitro human cadaver skin model has proven to be a valuable tool for the study of percutaneous absorption and the determination of the pharmacokinetics of topically applied drugs. The traditional model uses human cadaver skin mounted in specially designed diffusion chambers that allow the skin to be maintained at a temperature and humidity that match typical in vivo conditions. (Franz, T J: Percutaneous absorption: on the relevance of in vitro data. J. Invest. Dermatol., 1975, 64:190-195). This model has been modified to determine the pharmacokinetics of topical drugs applied to human cadaver fingernails. A finite dose (e.g. 10 μL/cm²) of formulation is applied to the outer surface of the fingernail and drug absorption is measured by monitoring its rate of appearance in the receptor solution bathing the inner surface of the fingernail. Data defining total absorption, rate of absorption, as well as fingernail content can be accurately determined in this model. The method is based on the human cadaver skin model, which has historic precedent for accurately predicting in vivo percutaneous absorption kinetics. (Franz T J: The finite dose technique as a valid in vitro model for the study of percutaneous absorption in man. In: Skin: Drug Application and Evaluation of Environmental Hazards, Current Problems in Dermatology, vol. 7, G. Simon, Z. Paster, M Klingberg, M. Kaye (Eds), Basel, Switzerland, S. Karger, 1978, pp 58-68.)

### Methods and Procedures:

Normal human cadaveric fingernails were obtained from four donors (three donors per formulation) and consisted of the first, second, third, and fourth fingernails. The fingernails were without obvious signs of disease or damage. At collection, the fingernails were sealed in a water-impermeable plastic bag, and stored at ≦−70° C. until the day of the experiment. Prior to use they were thawed at room temperature, cleared of any underlying tissue, and then rinsed in tap water to remove any adherent blood or other material from the surface.

Prior to being mounted the surface dimensions, weight and thickness of the fingernails were acquired and recorded. Fingernails from a single donor were fitted to 7 or 9 mm diameter modified Franz diffusion cells and secured in place with silicone sealant. The dermal chamber was filled to capacity with a receptor solution of phosphate-buffered isotonic saline (PBS), pH 7.4±0.1, and the epidermal chamber was left open to ambient laboratory environment. The cells were then placed in a water bath set to achieve a fingernail surface temperature of 32.0±1.0° C.

The test formulations outlined in Table 2 were evaluated in this study.

TABLE 2

| ID Number | Description |
|---|---|
| Formulation A | 10% omadine, 57% propylene glycol (PG), 33% |
| | isopropanol (IPA) |
| Formulation C | 10% omadine equivalent (as ammonium salt), 33% |
| | PG, 33% IPA, 24% water |
| Formulation D | 20% omadine equivalent (as ammonium salt) 33% |
| | PG, 47% water |
| Formulation F | 20% omadine equivalent (90% as ammonium salt), 20% |
| | PG, 18% IPA, 42% water |
| Formulation H | 20% omadine equivalent (80% as ammonium salt), 20% |
| | PG, 20% IPA, 40% water |

Prior to administration of the topical test formulations to the fingernail sections, a pre-dose sample was taken and the receptor solution was replaced with a fresh solution of 1× PBS.

Subsequently, each test product was applied to duplicate fingernail sections within the same donor. Pairs of nails from each donor received treatments such that each formulation was tested on three donors.

Dosing was performed using a positive displacement pipette set to deliver the appropriate amount to each fingernail to be equivalent to 10 μL/cm² of formulation. Each fingernail was dosed once per day for 14 consecutive days (24±3 hour intervals).

At 24 (±3) hour intervals, prior to dosing, the receptor solution was removed in its entirety, replaced with fresh receptor solution, and a predetermined volume aliquot saved for subsequent analysis.

After the last sample was collected (day 15), the fingernails were gently cleansed with a soap solution (1% Sodium Lauryl Sulfate in water), and water rinsed, using cotton-tipped swabs. The wash and swabs were discarded. Following the wash, the fingernails were removed from the chamber, minced, and extracted in equal parts methanol and water.

Quantification of Omadine was by High Performance Liquid Chromatography. Briefly, HPLC was conducted on a Hewlett-Packard 1100 Series HPLC system with a diode array detector. A solvent system consisting of A) 90% water with 0.1% TFA and B) 10% Acetonitrile with 0.1% TFA was run through a Phenomenex Gemini C18 column (50 mm×3.0 mm). Peak areas were quantified to concentration using an external standard curve prepared from the neat standard.

### Results:

Table 3 summarizes the results for the cumulative penetration of the various formulations through human cadaver fingernails over 14 days (Mean±SD, n=3 Donors) as well as the concentration of drug within the human cadaver fingernail at day 15 (Mean±SD, n=3 Donors).

TABLE 3

| | | Fingernail |
|---|---|---|
| | Cumulative Penetration | Concentration |
| Formulation | (μg/cm²) | (μg/mg) Day 15 |
| Formulation A | 1,605.6 ± 1,244.680 | 1.80 ± 0.99 |
| Formulation C | 2,119.3 ± 1,192.148 | 3.16 ± 1.00 |
| Formulation D | 3,542.1 ± 1,061.585 | 6.67 ± 2.19 |
| Formulation F | 3,920.1 ± 2,002.655 | 5.80 ± 5.66 |
| Formulation H | 4,410.9 ± 2,360.799 | 4.77 ± 4.57 |

The data indicate that omadine does penetrate into and through human fingernails from the five test formulations evaluated. The penetration profile demonstrates a continuous increase in the rate of penetration through the first six days of dosing, achieving a steady-state like rate of penetration thereafter.

Total mass absorbed, as characterized by cumulative penetration, rank orders the test formulations as: H>F>D>C>A.

Differences in penetration, as it relates to the test formulations, indicate that an increase in omadine concentration increases delivery (as would be predicted by Fick's law of diffusion). This is seen with formulation “H” (20%), F (20%), and “D” (20%)>formulation “C” (10%). The data also indicate that those formulations containing less of the ammonium salt of omadine (“F” and “H”) deliver more through the nails than those formulations consisting entirely of the ammonium salt of omadine (“D” and “C”) and the formulation containing only omadine (“A”). Finally, those formulations that contain water outperform the single formulation lacking water (“A”).

There were no statistically significant differences between the test formulations for total amount of Omadine penetration (p>0.02) using the Student's t-test. However, for nail omadine content, on study day 15, Formulations “D” and “H” both demonstrate a significantly greater nail concentration than Formulation “A” (p≦0.001), with the other comparisons not being significantly different.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A composition for topical administration of an antifungal agent, comprising an acidic antifungal agent with a molecular weight no greater than about 170 Daltons and/or a pharmaceutically acceptable salt thereof in an aqueous formulation having a pH no more than the pKa of the acidic antifungal agent plus about 2.5.

2. A composition for topical administration of an antifungal agent, comprising an acidic antifungal agent with a molecular weight no greater than about 170 Daltons in a substantially nonaqueous formulation, wherein said acidic antifungal agent optionally comprises a combination of the acidic antifungal agent and a pharmaceutically acceptable salt thereof, wherein the weight ratio of the pharmaceutically acceptable salt of the acidic antifungal agent to the acidic antifungal agent does not exceed 100:1.

3. The composition of claim 1 or 2, wherein the acidic antifungal agent comprises omadine, octanoic acid, sorbic acid, salicylic acid, hexanoic acid, or benzoic acid.

4. The composition of claim 1, wherein the pH of the composition is equal to about the pKa of the acidic antifungal agent.

5. The composition of claim 1, wherein the pH of the composition is no less than the pKa of the acidic antifungal agent minus about one.

6. The composition of claim 1 or 2, further comprising a glycol or glycerol.

7. The composition of claim 6, wherein the glycol comprises propylene glycol.

8. The composition of claim 1, wherein the acidic antifungal agent and/or pharmaceutically acceptable salt thereof comprises from about 0.1% to about 20% by weight of the composition.

9. The composition of claim 1 or 2, further comprising a film forming polymer.

10. The composition of claim 9, wherein the film forming polymer is selected from cellulose, cellulose derivatives, polyvinyl alcohol, or polyvinylpyrrolidone.

11. The composition of claim 10, wherein the film forming polymer comprises hydroxypropylcellulose.

12. The composition of claim 1 or 2, wherein the composition is formulated for delivery in the form of a lacquer, a gel, a patch, or a hydrating system.

13. The composition of claim 12, wherein the composition is formulated for delivery in the form of a patch, wherein the patch comprises an adhesive, and wherein the adhesive comprises the composition.

14. A composition for topical administration of an antifungal agent, comprising omadine and/or a pharmaceutically acceptable salt thereof, a glycol or glycerol, and an aqueous volatile solvent, wherein the pH of the composition is no more than the pKa of the omadine plus about 2.5.

15. The composition of claim 14, wherein the pharmaceutically acceptable salt of omadine comprises ammonium omadine.

16. The composition of claim 14, further comprising a film-forming polymer.

17. The composition of claim 16, wherein the film forming polymer comprises hydroxypropylcellulose.

18. The composition of claim 14, wherein the glycol comprises propylene glycol.

19. The composition of claim 14, wherein the volatile solvent comprises isopropanol or ethanol.

20. A composition for topical administration of an antifungal agent, comprising an ammonium salt of omadine.

21. A method of treating a nail fungal infection in an animal, comprising administering to an infected nail surface of an animal in need of such treatment a therapeutically effective amount of a composition of claim 1, 2, 14, or 20.

22. The method of claim 21, wherein the nail fungal infection is onychomycosis.

23. The method of claim 21, wherein the infected nail surface is a toenail.

24. The method of claim 21, wherein the animal is a human.

25. The method of claim 21, further comprising the conjoint administration of a second therapeutic agent capable of treating a fungal infection.

26. The method of claim 25, wherein the second therapeutic agent is 5-fluorocystine or terbinafine.
